(19) United States
(12) Patent Application Publication
(10) Pub. No.: US 2021/0346279 A1
(43) Pub. Date: Nov. 11, 2021

(54) COMPOSITIONS COMPRISING TAPINAROF FOR THE TREATMENT OF PRURITIS

(71) Applicant: Sol-Gel Technologies Ltd., Ness Ziona (IL)

(72) Inventors: Marcel ZIGHELBOIM, Kiryat Motzkin (IL); Moshe ARKIN, Kfar Shmaryahu (IL); Karine NEIMANN, Ness Ziona (IL); Hila HAKAK DJERBI, Tel Aviv (IL)

(73) Assignee: Sol-Gel Technologies Ltd., Ness Ziona (IL)

(21) Appl. No.: 17/313,136

(22) Filed: May 6, 2021

Related U.S. Application Data
(60) Provisional application No. 63/021,142, filed on May 7, 2020.
(60) Provisional application No. 63/128,219, filed on Dec. 21, 2020.

(51) Int. Cl. A61K 9/00; A61K 31/05; A61K 31/517; A61P 17/04; A61K 9/06

(52) U.S. Cl. CPC A61K 9/0014; A61K 31/05; A61K 9/06; A61P 17/04; A61K 31/517

(57) ABSTRACT

The present invention, in some embodiments thereof, relates to a topical composition for use in treating, preventing, alleviating and/or ameliorating pruritus, wherein the composition comprises tapinarof and the pruritus is not associated with atopic dermatitis, psoriasis or any combination thereof.

### CROSS REFERENCE TO RELATED APPLICATIONS

This application claims the benefit of U.S. Application Ser. No. 63/128,219 filed on Dec. 21, 2020, and U.S. Application Ser. No. 63/021,142 filed on May 7, 2020, which are all incorporated in their entirety herein by reference.

### FIELD OF THE INVENTION

The present invention, in some embodiments thereof, relates to a topical composition for use in treating, preventing and/or ameliorating pruritus, wherein the composition comprises tapinarof and the pruritus is not associated with atopic dermatitis, psoriasis or any combination thereof.

### BACKGROUND OF THE INVENTION

### Pruritus

Pruritus is defined as an unpleasant sensation of the skin leading to the desire to scratch (Stander, S. et al. Acta. Derm. Venereol. 2007; 87: 291-294). Pruritus can be distinguished as acute or chronic, with the latter defined as pruritus lasting 6 or more weeks according to a recommendation by the International Forum for the Study of Itch (IFSI). Chronic pruritus, which can be distressing and often refractory to treatment, is associated with many diseases. While many pruritic skin diseases can be diagnosed readily, this becomes more difficult for pruritus that occurs in apparently normal skin or accompanying secondary scratch-evoked lesions as a consequence of systemic disease. The quality, intensity and diurnal rhythm of itch are important factors that currently have limited diagnostic value.

In recent years fundamental new insights have been gained (Grundmann, S. et al. Ann Dermatol, 2011; Vol. 23, No. 1, 1-11) into pruritus induction and transmission as well as involved nerves, receptors and mediators. The understanding of the pathogenesis of pruritus has increased; the clinical assignment of the individual mechanism to the different pruritic diseases remains a task for future research.

### Granuloma Annulare (GA)

Granuloma annulare (GA) is a granulomatous, inflammatory skin disorder of unknown etiology. Granules, bumps, nodules and/or papules associated with this disorder usually burn or itch. While most often localized to the extremities and self-resolving, GA is generalized in 15% of those affected. Moreover, generalized and disseminated variants of GA often persist, is disfiguring, and can recur, causing many patients to seek treatment. Unfortunately, effective treatment options are limited, largely due to lack of pathogenic understanding of disease mechanism.

### Prurigo Nodularis (PN)

PN is characterized by hyperkeratotic excoriated papulonodular (itchy) lesions that show symmetrical distribution on the extensor surfaces of the body and/or extremities, the numbers of which range from several to hundreds (Akarsu, S. et al. An Bras Dermatol. 2018; 93(5):671-9). It is a rare disease and is one of the most difficult conditions in terms of determining its etiology and treatment among chronic skin diseases.

There is a need for a treatment to pruritus, especially pruritus which is not associated with atopic dermatitis or psoriasis.

### SUMMARY OF THE INVENTION

The present invention provides a method of treatment, prevention, alleviation and/or amelioration of pruritus, comprising administering topically to a subject in need thereof a therapeutically effective amount of a topical composition, wherein the composition comprises tapinarof and at least one topically acceptable solvent or carrier and/or excipient, and the pruritus is not associated with atopic dermatitis, psoriasis or any combination thereof.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 presents the number of scratches in a mouse model following treatment of AEW induced pruritus (as described in Example 2) using topical formulations of tapinarof (0.5%, 1% and 2% w/w). The number of scratches was summarized for each animal and the average number per group was calculated.

FIG. 2 presents the period of scratching time (seconds) during the evaluation hour of a mouse model following treatment of AEW induced pruritus (as described in Example 2) using topical formulations of tapinarof (0.5%, 1% and 2% w/w).

### DETAILED DESCRIPTION OF THE INVENTION

### Topical Compositions for Treating Pruritus

In one aspect, the present invention provides a method of treatment, prevention, alleviation and/or amelioration of pruritus, comprising administering topically to a subject in need thereof a therapeutically effective amount of a topical composition, wherein the composition comprises tapinarof and at least one topically acceptable solvent or carrier and/or excipient, and the pruritus is not associated with atopic dermatitis, psoriasis or any combination thereof.

In one aspect, the present invention provides a topical composition for use in treating, preventing and/or ameliorating pruritus, wherein the composition comprises tapinarof and the pruritus is not associated with atopic dermatitis, psoriasis or any combination thereof.

In one embodiment, the tapinarof is in an amount of about 0.1% w/w to about 10.0% w/w. In another embodiment, the tapinarof is in an amount of about 0.1% w/w to about 1.0% w/w. In another embodiment, the tapinarof is in an amount of about 0.1% w/w to about 2.0% w/w. In another embodiment, the tapinarof is in an amount of about 1.0% w/w to about 2.0% w/w. In another embodiment, the tapinarof is in an amount of about 2.0% w/w to about 5.0% w/w. In another embodiment, the tapinarof is in an amount of about 5.0% w/w to about 10.0% w/w. In another embodiment, the tapinarof is in an amount of 0.1%, 0.2%, 0.3%, 0.4%, 0.5%, 0.6%, 0.7%, 0.8%, 0.9%, 1%, 1.5%, 2%, 4%, 5% or 7% w/w. In another embodiment, the tapinarof is in an amount of 0.5% w/w. Each possibility represents a separate embodiment of the present invention.

In one embodiment, the topical composition of the present invention further comprises at least one additional active agent selected from an Epidermal Growth Factor Receptor (EGFR) inhibitor, a corticosteroid, calcipotriene, a Janus kinase inhibitor (JAK inhibitor), a phosphodiesterase-4 inhibitor (PDE4 inhibitor), a calcineurin inhibitor, an antibiotic, a cannabinoid receptor agonist, a kappa-opioid receptor agonist, and agents that target thymic stromal lymphopoietin (TSLP) like Tezepelumab or an Antibody and combinations thereof, in a concentration of from about 0.01% to about 1%, from about 1% to about 3%, from about 3% to about 5% w/w. In another embodiment, the EGFR inhibitor is erlotinib, gefitinib, lapatinib, cetuximab, panitumumab, vandetanib, necitumumab, osimertinib or any combination thereof. In another embodiment, the EGFR inhibitor is erlotinib. In another embodiment, the corticosteroid is hydrocortisone, budesonide, cortisone, betamethasone, mometasone, salts thereof or any combination thereof. In another embodiment, the JAK inhibitor is tofacitinib, abrocitinib, ruxolitinib, delgocitinib, oclacitinib, baricitinib, peficitinib or any combination thereof. In another embodiment, the PDE4 inhibitor is selected from roflumilast, roflumilast N-oxide, apremilast, piclamilast, ibudliast, cilomilast their salts, hydrates or solvates and combinations thereof. In another embodiment, the calcineurin inhibitor is tacrolimus, pimecrolimus, ciclosporin, voclosporin or any combination thereof. In another embodiment, the antibiotic is imidazole (e.g. metronidazole, tinidazole), triazole (e.g. fluconazole, cefatrizine) or tetrazole (e.g. cefmetazole, cefazoline) based antibiotic or any combination thereof. In another embodiment, the antibody is Omalizumab for it-4, Dupilumab for IL-4/IL-13, Lebrikizumab and Tralokinumab for IL-13, Nemolizumab for IL-31, Ustekinumab for IL-12/23, Fezakinumab for IL-22, Adalimumab, Cetrolizumab, Inflixiab and Etanercept for TNFa, Seckukinumab, Brodalumab and Ixekizumab for IL-17a, Guselkumab, Tildakizumab and Risankisunab for IL-23, Ustekinumab for IL-12/23, or any combination thereof. Each possibility represents a separate embodiment of the present invention.

In one embodiment, the topical composition of the present invention further comprises at least one penetration enhancer. In another embodiment, the penetration enhancer is in a concentration of between 10% w/w to about 98% w/w of said composition. In another embodiment, the penetration enhancer is in a concentration of between 10% w/w to about 20% w/w of said composition. In another embodiment, the penetration enhancer is in a concentration of between 20% w/w to about 30% w/w of said composition. In another embodiment, the penetration enhancer is in a concentration of between 30% w/w to about 40% w/w of said composition. In another embodiment, the penetration enhancer is in a concentration of between 40% w/w to about 50% w/w of said composition. In another embodiment, the penetration enhancer is in a concentration of between 50% w/w to about 60% w/w of said composition. In another embodiment, the penetration enhancer is in a concentration of between 60% w/w to about 70% w/w of said composition. In another embodiment, the penetration enhancer is in a concentration of between 70% w/w to about 80% w/w of said composition. In another embodiment, the penetration enhancer is in a concentration of between 80% w/w to about 90% w/w of said composition. In another embodiment, the penetration enhancer is in a concentration of between 90% w/w to about 98% w/w of said composition. In another embodiment, the penetration enhancer is selected from dimethyl sulfoxide (DMSO), ethanol, isopropyl alcohol, dimethyl isosorbide, isopropyl myristate, oleic acid, a polyethylene glycol, hexylene glycol, glycofurol and combinations thereof. Each possibility represents a separate embodiment of the present invention.

In one embodiment, the topical composition of the present invention further comprises at least one topically acceptable carrier or solvent. In another embodiment, the at least one topically acceptable carrier or solvent is selected from dimethyl sulfoxide (DMSO), ethanol, isopropyl alcohol, propylene glycol, dimethyl isosorbide, isopropyl myristate, oleic acid, a polyethylene glycol, hexylene glycol, glycerin, glycofurol and combinations thereof. Each possibility represents a separate embodiment of the present invention.

In another embodiment, the at least one penetration enhancer has dual functionality and may act also as a solvent or a carrier.

In one embodiment, the topical composition of the present invention further comprises a topically acceptable excipient. In another embodiment, the topically acceptable excipient is selected from a gelling agent, a conditioner, an emulsifier, an emollient, a preservative or any combination thereof. Each possibility represents a separate embodiment of the present invention.

In one embodiment, non-limiting examples of the gelling agent include: acrylic acid polymer, polysaccharides, gelatin, alginate, pectin and any combination thereof. In another embodiment, the gelling agent is an acrylic acid polymer. Each possibility represents a separate embodiment of the present invention.

In one embodiment, non-limiting examples of the emulsifier include: sodium lauryl ether sulfate, disodium laureth sulfosuccinate, mixture of sodium lauryl ether sulfate and disodium laureth sulfosuccinate, polysorbate 20, cetrimonium bromide, Triton-X-100, glycerol monostearate and any combination thereof. In another embodiment, the emulsifier is a mixture of sodium lauryl ether sulfate and disodium laureth sulfosuccinate. Each possibility represents a separate embodiment of the present invention.

In one embodiment, non-limiting examples of the conditioner include: polyquaternium-10, oleyl alcohol, cetyl alcohol and any combination thereof. In another embodiment, the conditioner is polyquaternium-10. Each possibility represents a separate embodiment of the present invention.

In one embodiment, non-limiting examples of the emollient include: coconut fatty acid diethanolamide, petrolatum, castor oil and any combination thereof. In another embodiment, the emollient is coconut fatty acid diethanolamide Each possibility represents a separate embodiment of the present invention.

In one embodiment, non-limiting examples of the preservative include: imidurea, sorbic acid, parabens, ascorbic acid, gallic acid and any combination thereof. In another embodiment, the preservative is imidurea. Each possibility represents a separate embodiment of the present invention.

In one embodiment, the topical composition of the present invention is in a dosage form selected from a shampoo, a cream, a lotion, a gel, an ointment, an emulsion, a solution, a suspension, an elixir, a tincture, a paste, a foam, an aerosol, a spray, a roll-on applicator, a patch and an applicator syringe. Each possibility represents a separate embodiment of the present invention.

In one embodiment, the topical composition of the present invention further comprises at least one topically acceptable carrier or solvent and/or excipient.

In some embodiments, the tapinarof of the composition of the present invention is encapsulated or non-encapsulated. In one embodiment, the composition of the present invention comprises tapinarof and at least one additional active agent as described hereinabove. In another embodiment, both the tapinarof and the at least one additional active agent are encapsulated or non-encapsulated. In another embodiment, the tapinarof or the additional active agent is encapsulated. In another embodiment, tapinarof and the additional active agent are both encapsulated in the same capsule. In another embodiment, tapinarof and the additional active agent are both encapsulated in different capsules. In some embodiments, tapinarof and/or the additional active agent are encapsulated in a microcapsule, where “microcapsule” refers to a microparticle having a core shell structure, wherein said core comprises an active agent as defined herein (e.g. tapinarof and/or an additional active agent such as Erlotinib), being coated by a shell forming the microcapsule entrapping the core. Each possibility represents a separate embodiment of the present invention.

In further embodiments, the compositions of the present invention for treating pruritus (not associated with atopic dermatitis and/or psoriasis) are controlled or slowed release drug delivery systems, wherein the active agent(s) is encapsulated, coated, adsorbed, embedded, impregnated, dispersed, entrapped, or encased in a polymeric material and providing a sustained release formulation. Each possibility represents a separate embodiment of the present invention.

According to some embodiments of the present invention, the coated form of the active agent(s) (microcapsule) may be in form of a polymeric microsponge/silica microsphere where the tapinarof is adsorbed, embedded, impregnated or entrapped in the microsponge/silica microsphere as described for example in U.S. Pat. Nos. 4,690,825; 5,145,675, 5,879,716, 5,955,109, and 9,452,137 incorporated herein by reference in their entirety. Each possibility represents a separate embodiment of the present invention.

In other embodiments, microcapsules are formed by the encapsulation process disclosed in the following publications (herein incorporated by reference): U.S. Pat. Nos. 7,629,394, 9,205,395, US 2015/0328615, US 2014/0186630. Controlled release microcapsules: IN01958CH2007, IN02080CH2007, U.S. Pat. Nos. 4,235,872, 4,670,250, EP 0248531, U.S. Pat. Nos. 4,970,031, 5,238,714, WO9321764, U.S. Pat. No. 5,575,987, WO9420075, US 2004/137031, US 2006/003014, US 2010/180464. Each possibility represents a separate embodiment of the present invention.

When referring to a “controlled or slowed release drug delivery system” it should be understood to relate to a delivery system (which in the present invention is a topical delivery system) that enables the release of the pharmaceutical active agent in predetermined amounts over a specified period. In some embodiments said system is a core-shell system of a microcapsule or a porous matrix structure, such as for example a microsponge.

The term “embedded” as used herein should be understood to encompass an inert system that provides a barrier between the pharmaceutical active agent (e.g. tapinarof and/or an additional active agent such as erlotinib) and its surrounding environment in the composition. In some embodiments said agent(s) is entrapped and/or encapsulated in said controlled release system.

### Methods of Treatment

In one further aspect, the present invention provides a method of treatment, prevention, alleviation and/or amelioration of pruritus, comprising administering topically to a subject in need thereof a therapeutically effective amount of a composition as described hereinabove, wherein the composition comprises tapinarof and the pruritus is not associated with atopic dermatitis, psoriasis or any combination thereof. In one specific aspect, the method of treatment, prevention, alleviation and/or amelioration of pruritus as described above further comprises treating the subject with photoirradiation. In one embodiment, the composition to be administered topically in the methods of the present invention further comprises at least one topically acceptable carrier or solvent and/or excipient.

In one further aspect, the present invention provides a method of treatment, prevention, alleviation and/or amelioration of pruritus, wherein the pruritus is not associated with atopic dermatitis, psoriasis or any combination thereof, comprising administering topically to a subject in need thereof from about 0.1 to about 10% w/w, from about 0.1% to about 2% w/w, from about 1 to about 5% w/w, from about 3 to about 7% w/w of tapinarof and at least one additional active agent selected from an Epidermal Growth Factor Receptor (EGFR) inhibitor, a corticosteroid, calcipotriene, a Janus kinase inhibitor (JAK inhibitor), a phosphodiesterase-4 inhibitor (PDE4 inhibitor), a calcineurin inhibitor, an antibiotic, a cannabinoid receptor agonist, a kappa-opioid receptor agonist, and agents that target thymic stromal lymphopoietin (TSLP) like Tezepelumab or an antibody and combinations thereof, in a concentration of from about 0.01% to about 1%, from about 1% to about 3%, from about 3% to about 5% w/w. In another embodiment, the EGFR inhibitor is erlotinib, gefitinib, lapatinib, cetuximab, panitumumab, vandetanib, necitumumab, osimertinib or any combination thereof. In another embodiment, the EGFR inhibitor is erlotinib. In another embodiment, the corticosteroid is hydrocortisone, budesonide, cortisone, betamethasone, mometasone, salts thereof or any combination thereof. In another embodiment, the JAK inhibitor is tofacitinib, abrocitinib, ruxolitinib, delgocitinib, oclacitinib, baricitinib, peficitinib or any combination thereof. In another embodiment, the PDE4 inhibitor is selected from roflumilast, roflumilast N-oxide, apremilast, piclamilast, ibudliast, cilomilast their salts, hydrates or solvates and combinations thereof. In another embodiment, the calcineurin inhibitor is tacrolimus, pimecrolimus, ciclosporin, voclosporin or any combination thereof. In another embodiment, the antibiotic is imidazole (e.g. metronidazole, tinidazole), triazole (e.g. fluconazole, cefatrizine) or tetrazole (e.g. cefmetazole, cefazoline) based antibiotic or any combination thereof. In another embodiment, the antibody is Omalizumab for it-4, Dupilumab for IL-4/IL-13, Lebrikizumab and Tralokinumab for IL-13, Nemolizumab for IL-31, Ustekinumab for IL-12/23, Fezakinumab for IL-22, Adalimumab, Cetrolizumab, Inflixiab and Etanercept for TNFa, Seckukinumab, Brodalumab and Ixekizumab for IL-17a, Guselkumab, Tildakizumab and Risankisunab for IL-23, Ustekinumab for IL-12/23, or any combination thereof.

In one further aspect, the present invention provides a method of treatment, prevention, alleviation and/or amelioration of pruritus wherein the pruritus is not associated with atopic dermatitis, psoriasis or any combination thereof, comprising administering topically to a subject in need thereof from about 0.1 to about 2% w/w of tapinarof and at least one additional active agent selected from an Epidermal Growth Factor Receptor (EGFR) inhibitor, a corticosteroid, calcipotriene, a Janus kinase inhibitor (JAK inhibitor), a phosphodiesterase-4 inhibitor (PDE4 inhibitor), a calcineurin inhibitor, an antibiotic, a cannabinoid receptor agonist, a kappa-opioid receptor agonist, and agents that target thymic stromal lymphopoietin (TSLP) like Tezepelumab or an antibody and combinations thereof, in a concentration of from about 0.01% to about 1%, from about 1% to about 3%, from about 3% to about 5% w/w. In another embodiment, the EGFR inhibitor is erlotinib, gefitinib, lapatinib, cetuximab, panitumumab, vandetanib, necitumumab, osimertinib or any combination thereof. In another embodiment, the EGFR inhibitor is erlotinib. In another embodiment, the corticosteroid is hydrocortisone, budesonide, cortisone, betamethasone, mometasone, salts thereof or any combination thereof. In another embodiment, the JAK inhibitor is tofacitinib, abrocitinib, ruxolitinib, delgocitinib, oclacitinib, baricitinib, peficitinib or any combination thereof. In another embodiment, the PDE4 inhibitor is selected from roflumilast N-oxide, roflumilast, apremilast, piclamilast, ibudliast, cilomilast their salts, hydrates or solvates and combinations thereof. In another embodiment, the calcineurin inhibitor is tacrolimus, pimecrolimus, ciclosporin, voclosporin or any combination thereof. In another embodiment, the antibiotic is imidazole (e.g. metronidazole, tinidazole), triazole (e.g. fluconazole, cefatrizine) or tetrazole (e.g. cefmetazole, cefazoline) based antibiotic or any combination thereof. In another embodiment, the antibody is Omalizumab for il-4, Dupilumab for IL-4/IL-13, Lebrikizumab and Tralokinumab for IL-13, Nemolizumab for IL-31, Ustekinumab for IL-12/23, Fezakinumab for IL-22, Adalimumab, Cetrolizumab, Inflixiab and Etanercept for TNFa, Seckukinumab, Brodalumab and Ixekizumab for IL-17a, Guselkumab, Tildakizumab and Risankisunab for IL-23, Ustekinumab for IL-12/23, or any combination thereof. In another embodiment, 0.5% w/w tapinarof is administered.

According to an aspect of the invention, there is provided a method of treatment, prevention or amelioration of pruritus wherein the pruritus is not associated with atopic dermatitis, psoriasis or any combination thereof, which is treatable, preventable, ameliorable and/or alleviated by treatment of a subject in need thereof with a combination composition of tapinarof and at least one additional active agent selected, the method comprising co-administering to a subject in need thereof therapeutically effective amounts of tapinarof and at least one additional active agent, thereby treating, curing or alleviating the pruritus.

In some embodiments, the effective amount is a therapeutically effective amount of tapinarof and at least one additional active agent, namely an amount which will cure, treat, mitigate or prevent pruritus.

In some embodiments, co-administration of tapinarof and at least one additional active agent exhibits an additive or synergistic effect while treating or alleviating a skin disorder.

In some other embodiments, the co-administration may be made either by administration of a single combination composition, or alternatively by separate administration of a first composition comprising tapinarof and a second composition comprising the at least one additional active agent.

In one embodiment, the method comprises once daily or twice daily topical administration of the composition.

In one embodiment, tapinarof is administered within the method in amounts as described hereinabove, e.g. between 0.1% and 10% w/w or 1%, 2%, 4%, 5% or 7% w/w.

In one embodiment, tapinarof is administered within the method in amounts as described hereinabove, e.g. between 0.1% and 2% w/w or 0.1%, 0.2%, 0.3%, 0.4%, 0.5%, 0.6%, 0.7%, 0.8%, 0.9%, 1%, 1,5%, 2%, 4%, 5% or 7% w/w.

In some embodiments, the pruritus within the composition or method of the present invention is chronic pruritus. In one embodiment, the pruritus is of unknown or undetermined origin (PUO). In one other embodiment, the pruritus is associated with a skin or subcutaneous tissue disease or condition, a systemic disease or condition, a neurological, psychiatric or psychosomatic disease or condition, or any combination thereof. In one further embodiment, the pruritus is associated with granuloma annulare or prurigo nodularis. Each possibility represents a separate embodiment of the present invention.

### Regimen of Administration

Therapeutically effective concentrations of tapinarof (and optionally at least one additional active agent) in the compositions of the present invention for treatment, prevention, alleviation and/or amelioration of pruritus, are determined by empirical methods known in the art.

The concentration of the active agents in the composition will depend on absorption, inactivation, excretion rates of the active compound, synergistic and/or additive effects, the dosage schedule, and amount administered as well as other factors known to those of skill in the art. The dosage and regimen of administration may be determined by dose finding studies, as known in the art.

Exemplary dosages, strengths and concentrations of tapinarof in the compositions administered topically, are about 0.1%, 0.25%, 0.5%, 1%, 2%, 3%, 4%, 5%, 6%, 7%, 8%, 9% or 10% w/w. Typical strengths in the topical combination compositions of the present invention are 0.2%, 0.3%, 0.4%, 0.5%, 0.6%, 0.7%, 0.8%, 0.9%, 1%, 1.5%, 2%, 4%, 5% or 7% w/w of tapinarof. Each possibility represents a separate embodiment of the present invention.

Exemplary dosages, strengths and concentrations of the at least one additional active agent in the compositions administered topically, are from about 0.01% to about 1%, from about 1% to about 3%, from about 3% to about 5% w/w, about 0.05%, 0.1%, 0.25%, 0.5%, 1%, 2%, 3%, 4%, 5%, 6%, 7%, 8%, 9%, 10%, 12%, 15% or about 20% w/w. Each possibility represents a separate embodiment of the present invention.

The frequency of administration can be determined empirically. Exemplary frequencies are once daily, twice daily, weekly, bi-weekly or monthly. Each possibility represents a separate embodiment of the present invention.

Typical administration frequencies of the topical compositions of the present invention are once daily and twice daily.

Dosage frequencies can be gradually attenuated over time and maintained at a steady dose suitable for long-term—six months, 1 year, 5 years, 10 years or more, up to lifelong administration to control the symptoms of the pruritus. For example, dosage administration can begin at from twice a day, to once a day, to two times a week, to once a week, to once every two weeks or less frequent than once every two weeks. Each possibility represents a separate embodiment of the present invention.

### Kits

There are provided kits containing the compositions of the present invention, optionally including instructions for administration. The combinations include, for example, the compositions as provided herein. Additionally, provided herein are kits containing the above-described combinations and optionally instructions for administration by topical, or other routes, depending on the composition to be delivered.

The compositions provided herein can be packaged as articles of manufacture containing packaging material, a composition provided herein, and a label that indicates that the composition is for treating pruritus, and is formulated for topical delivery.

The articles of manufacture provided herein contain packaging materials. Packaging materials for use in packaging pharmaceutical products are well known to those of skill in the art. Examples of pharmaceutical packaging materials include, but are not limited to bottles, tubes, containers, application syringes and any packaging material suitable for a selected formulation and intended mode of administration and treatment.

### Definitions

Unless defined otherwise, all technical and scientific terms used herein have the same meaning as is commonly understood by one of skill in the art to which the invention pertains. In case of conflict, the specification, including definitions, takes precedence. All patents, patent applications, published applications, articles, publications and other published materials referred to throughout the entire disclosure herein, unless noted otherwise, are incorporated by reference in their entirety.

As used herein, the indefinite articles “a” and “an” mean “at least one” or “one or more” unless the context clearly dictates otherwise.

As used herein, the term “treating” or “treatment” includes curing a condition, treating a condition, preventing a condition, treating symptoms of a condition, curing symptoms of a condition, ameliorating symptoms of a condition, treating effects of a condition, ameliorating effects of a condition, and preventing results of a condition.

As used herein, the terms “pharmaceutically active agent” or “active agent” or “active pharmaceutical ingredient” or “API” are interchangeable and mean the ingredient is a pharmaceutical drug which is biological active and is regulatory approved or approvable as such.

The term “ingredient” refers to a pharmaceutically acceptable ingredient which is included or is amenable to be included in FDA's Inactive Ingredient database (IIG). Inactive ingredients sometimes exhibit some therapeutic effects, although they are not drugs.

As used herein, a “pharmaceutical composition” refers to a composition comprising one or more active ingredients with other components such as pharmaceutically acceptable ingredients or excipients. The purpose of a pharmaceutical composition is to facilitate administration of an active ingredient to a subject. In one embodiment, the ingredients and/or excipients are topically acceptable.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 μm” is intended to mean “about 10 μm”.

As used herein, numerical ranges preceded by the term “about” should not be considered to be limited to the recited range. Rather, numerical ranges preceded by the term “about” should be understood to include a range accepted by those skilled in the art for any given element in formulations according to the present invention.

As used herein, when a numerical value is preceded by the term “about”, the term “about” is intended to indicate +/−10%.

The terms “comprise”, “comprising”, “includes”, “including”, “having” and their conjugates mean “including but not limited to”.

The term “consisting of” means “including and limited to”.

The term “consisting essentially of” means that the composition, method formulation may include additional ingredients, steps and/or parts, but only if the additional ingredients, steps and/or parts do not materially alter the basic and novel characteristics of the claimed composition, method or structure.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include chemical, molecular and biochemical, techniques. Such techniques are thoroughly explained in the literature. General references are provided throughout this document. The procedures therein are believed to be well known in the art and are provided for the convenience of the reader. All the information contained therein is incorporated herein by reference.

### Example 1

Preparation of tapinarof, 1% lotion

| Raw Material (compendial Name) | % w/w |
|---|---|
| Tapinarof 98% | 1.0 |
| Castor oil | 4.0 |
| Mineral oil light | 4.0 |
| Diethylene glycol monoethyl ether | 5.5 |
| Dimethyl Sulfoxide | 5.5 |
| Sorbitan Monooleate | 0.1 |
| Propylene glycol | 10.0 |
| Disodium Edetate (EDTA) | 0.1 |
| Carbomer Copolymer Type B | 0.4 |
| Pemulen ®TR-1 | |
| Carbomer Homopolymer Type A | 0.6 |
| Carbopol ®0981 | |
| Purified water | 64.00 |
| Benzoic Acid | 0.25 |
| BHT | 0.1 |
| Citric Acid | 0.1 |
| Sodium Citrate | 0.2 |
| Sodium hydroxide pellets | For pH adjustment |
| Purified water | q.s. to 100 |

### Water Phase

Into a glass beaker water and Benzoic Acid were added. The beaker was placed inside a hot water bath adjusted to 60° C. and the mixture was mixed with a magnetic stirrer until a clear solution free from particles was obtained. Then EDTA, Citric Acid and Sodium Citrate were added. The mixing was continued until a clear solution was obtained. The solution was cooled down to room temperature. Then, the pH was slowly adjusted to pH 6.0 with NaOH 20%.

### Oil Phase

In a separate glass beaker Mineral oil light, castor oil, span 80 and BHT were weighed. The beaker was placed inside a hot water bath adjusted to 60° C. and the mixture was mixed with a magnetic stirrer until a uniform solution was obtained. Then Carbopol®981 and Pemulen®TR-1 were slowly added and the mixing was continued until a homogenous mixture was obtained. The mixture was cooled down to room temperature.

### Active Phase

Into a separate glass beaker Propylene Glycol, Transcutol and DMSO were weighed. The mixture was mixed with a magnetic stirrer until a uniform homogenous solution was obtained. The beaker was covered with an aluminum foil and placed in a yellow light hood. Tapinarof was slowly added, and the mixing was continued for about 1 h until a clear solution free from particles was obtained.

The oil phase was slowly added to the water phase while homogenizing for about 5 minutes, until there were no lumps. Then, the active phase was slowly added to the Water+Oil phase while homogenizing for about 5 minutes.

Water was added for batch completion, and final pH was measured to conform it is around pH 5.

### Example 2

Tapinarof for Treating Pruritus

### Objective

The objective of this study was to examine the efficacy of topical formulations of Tapinarof in the strength of 0.5%, 1% and 2% as a treatment in acetone-ether-water (AEW) induced pruritus in a mouse model.

### Animal Study Procedure

The model of inducing itching sensation was done by treating C57/b16 mice with AEW as was conducted in other studies in the past.

The mice were shaved 24 h prior to study to expose the skin. For 5 days, twice a day (at least 3-4 hours apart), mice were applied acetone and ether (mixed at 1:1 ratio) on the right cheek for 30 seconds, and immediately afterwards applied on cheek water for 30 seconds.

Day 1-3: AEW was treated twice (at least 3-4 hours apart), AE 30s, Water 30s.

Day 4-5: AEW was treated twice (at least 3-4 hours apart), AE 30s, Water 30s, topical administration of vehicle, tapinarof 0.5%, tapinarof 1%, tapinarof 2% (−20 h before evaluation) and randomization.

Day 5: AEW was treated once, topical administration of vehicle, tapinarof 0.5%, tapinarof 1%, and tapinarof 2% (−2 h before evaluation), positive control dosing (−30 min), video record (evaluation), animal weighing, sacrificing, plasma collection and skin sample collection.

Immediately after AEW application on day 5, the mice were put into acrylic chambers and behavior was recorded for 60 min using a video camera under unmanned conditions to assess spontaneous scratching. Videos were manually scored for time spent spontaneously scratching the affected cheek. Scratching is defined as an episode in which the mouse lifted its paw and scratched continuously for any length of time until the paw returned to the floor. Only scratches of the affected cheek with their hind paws were counted. Other scratching behaviors were disregarded. The number of scratches was summarized for each animal and the average number per group was calculated. In addition, the scratching time (seconds) was also evaluated, and the average of scratching time was calculated. Data are expressed as the mean±S.E.M. statistically significant differences between two groups were evaluated using paired or non-paired Student's t-test as required.

### Results

### Animal Weighs

Animals weighed at baseline and at the time of sacrifice. No significance changes were reported between baseline and the end of study.

### Clinical Score

Behavioral scoring was performed by an observe blinded to the experimental condition.

### Number of Scratch Sessions

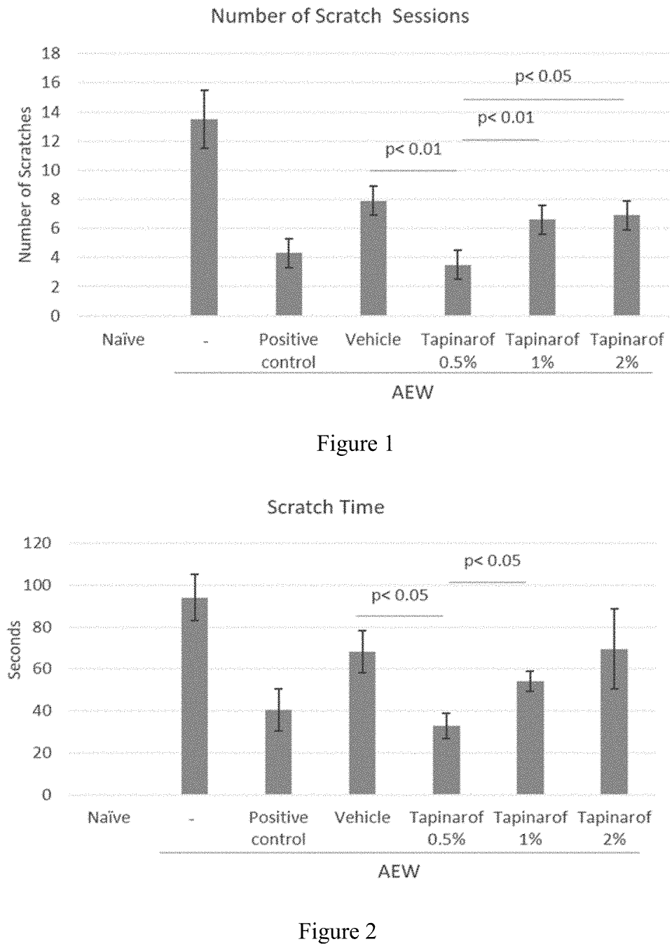

The number of scratches was summarized for each animal and the average number per group was calculated (FIG. 1).

### Scratches Time

Scratching time (seconds) was also evaluated, and the average of scratching time was calculated. (FIG. 2). Naïve group showed no scratching. The AEW group with no treatment presented 13 scratches sessions and an average of 94 seconds of scratching during the evaluation hour. The positive control has decreased the scratching time in ˜65% to 32 seconds and the sessions to 4.

The tapinarof 0.5% had a major effect over the vehicle (˜40%) and comparable to the positive control. Unexpectedly, increasing tapinarof percent twice and four times did not reduce the scratching sessions and periods but even increased it like or close to the vehicle effect.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method of treatment, prevention, alleviation and/or amelioration of pruritus, comprising administering topically to a subject in need thereof a therapeutically effective amount of a topical composition, wherein the composition comprises tapinarof and at least one topically acceptable solvent or carrier and/or excipient, and the pruritus is not associated with atopic dermatitis, psoriasis or any combination thereof.

2. The method of claim 1, wherein the tapinarof is in an amount of about 0.1% w/w to about 2.0% w/w.

3. The method of claim 2, wherein the tapinarof is in an amount of 0.5% w/w.

4. The method of claim 1, wherein the method comprises further administering at least one additional active agent selected from an Epidermal Growth Factor Receptor (EGFR) inhibitor, a corticosteroid, calcipotriene, a Janus kinase inhibitor (JAK inhibitor), a phosphodiesterase-4 inhibitor (PDE4 inhibitor), a calcineurin inhibitor, an antibiotic, a cannabinoid receptor agonist, a kappa-opioid receptor agonist, and agents that target thymic stromal lymphopoietin (TSLP) like Tezepelumab, an antibody and combinations thereof, in a concentration of from about 0.01% to about 1%, from about 1% to about 3%, from about 3% to about 5% w/w.

5. The method of claim 4, wherein said EGFR inhibitor is erlotinib, gefitinib, lapatinib, cetuximab, panitumumab, vandetanib, necitumumab, osimertinib or any combination thereof.

6. The method of claim 5, wherein said EGFR inhibitor is erlotinib.

7. The method of claim 1, wherein the composition further comprises at least one penetration enhancer.

8. The method of claim 7, wherein the penetration enhancer is in a concentration of between 10% w/w to about 98% w/w of said composition.

9. The method of claim 7, wherein the at least one penetration enhancer is selected from dimethyl sulfoxide (DMSO), ethanol, isopropyl alcohol, dimethyl isosorbide, isopropyl myristate, oleic acid, a polyethylene glycol, hexylene glycol, glycofurol and combinations thereof.

10. The method of claim 8, wherein the at least one penetration enhancer is selected from dimethyl sulfoxide (DMSO), ethanol, isopropyl alcohol, dimethyl isosorbide, isopropyl myristate, oleic acid, a polyethylene glycol, hexylene glycol, glycofurol and combinations thereof.

11. The method of claim 1, wherein the at least one solvent or carrier is selected from dimethyl sulfoxide (DMSO), ethanol, isopropyl alcohol, propylene glycol, dimethyl isosorbide, isopropyl myristate, oleic acid, a polyethylene glycol, hexylene glycol, glycerin, glycofurol and combinations thereof.

12. The method of claim 9, wherein the at least one penetration enhancer has dual functionality and may act also as a solvent or carrier.

13. The method of claim 1, wherein the composition further comprises an excipient.

14. The method of claim 13, wherein the excipient is selected from a gelling agent, a conditioner, an emulsifier, an emollient, a preservative or any combination thereof.

15. The method of claim 1, wherein said tapinarof is encapsulated or non-encapsulated.

16. The method of claim 1, wherein the composition is in a dosage form selected from a shampoo, a cream, a lotion, a gel, an ointment, an emulsion, a solution, a suspension, an elixir, a tincture, a paste, a foam, an aerosol, a spray, a roll-on applicator, a patch and an applicator syringe.

17. The method of claim 1, wherein the pruritus is chronic pruritus.

18. The method of claim 1, wherein the pruritus is of unknown or undetermined origin (PUO).

19. The method of claim 1, wherein the pruritus is associated with a skin or subcutaneous tissue disease or condition, a systemic disease or condition, a neurological, psychiatric or psychosomatic disease or condition, or any combination thereof.

20. The method of claim 1, wherein the pruritus is associated with granuloma annulare or prurigo nodularis.

21. The method of claim 1 wherein the method comprises once daily or twice daily topical administration of the composition.

22. The method of claim 1, further comprising treating the subject with photoirradiation.
